(19) United States
(12) Patent Application Publication
(10) Pub. No.: US 2012/0191082 A1
(43) Pub. Date: Jul. 26, 2012

(54) Method And Apparatus For Regulating The Formation Of Ice On A Catheter

(75) Inventor: H. Toby Markowitz, Roseville, MN (US)

(73) Assignee: Medtronic, Inc.

(21) Appl. No.: 13/440,142

(22) Filed: Apr. 5, 2012

Related U.S. Application Data
(63) Continuation of application No. 13/013,030, filed on Jan. 25, 2011.
Application No. 13/013,030 is a continuation of application No. 13/013,042, filed on Jan. 25, 2011.

(51) Int. Cl. A61B 18/02

(52) U.S. Cl. 606/21

(57) ABSTRACT

A method and apparatus is provided, including regulating the generation and formation of ice on an elongate body applied to the tissue of a patient. The method may include measuring a parameter from a sensor disposed on the instrument. The forming of ice and active warming of the instrument may be via a thermal source in fluid communication with the instrument.

### CROSS-REFERENCE TO RELATED APPLICATIONS

This application is a continuation of co-pending U.S. patent application Ser. No. 13/013,030, filed Jan. 25, 2011, and co-pending U.S. patent application Ser. No. 13/013,042, filed Jan. 25, 2011.

### FIELD OF THE INVENTION

This document relates generally to a medical device and more particularly to a method and apparatus to regulate the cooling and active warming of an instrument in the heart of a patient.

### BACKGROUND

Cryogenics may be used in medical applications to temporarily attach to and stabilize an instrument to a tissue and to modify properties of tissue. The tools used for the cryogenic application may range from a canister of very cold substance applied through a simple nozzle as in dermatologic applications, to sophisticated catheters placed within the body. When used within the body, an external source of an extremely cold coolant may be supplied to an instrument, the coolant circulated through the instrument and the coolant distributed to an applicator to affect a targeted tissue. U.S. Pat No. 5,147,355 issued to Friedman, et al. discloses a catheter having a fluid flow passage for directing a flow of cryogenic fluid to the tip of the catheter.

The cooling of the tissue surrounding the applicator effects change in the tissue depending on the degree to which the tissue is cooled and the duration of the cooling. Cooling the applicator below the freezing point of water (zero degrees Celsius) causes ice to form on the applicator and in the tissue surrounding it. Cryoablation catheters are used for the purposeful and therapeutic modification of specific tissues within the body. Such catheters are used, for example, to treat cardiac arrhythmias. The catheters are placed within the heart and are then used cryogenically to modify the propagation of depolarization within the heart. U.S. Pat. No. 7,357,797 issued to Ryba discusses a cryoablation catheter with a cooled tip and a temperature sensor positioned in a chamber at the distal tip of the catheter.

Cooling a metal applicator below the freezing point of water, zero degrees Celsius, may cause the applicator to adhere to the tissue. Cooling the applicator to around minus 30 degrees Celsius, and applying the applicator to the heart results in reversible changes to the myocardium. U.S. Pat No. 5,733,280 issued to Avitall describes the cooling of cardiac cells and rewarming the tissue resulting in total recovery of the tissue without damage. Cooling the applicator to around minus 60 degrees Celsius, however, can result in permanent change to the tissue.

As described above, measurement of the temperature of the applicator is commonly practiced by placing a temperature sensor in or on the applicator. The temperature in the core of the human body during normal physiologic conditions is about plus 37 degrees Celsius. Cooling the applicator causes a significant temperature gradient between the chamber where the coolant is applied in the applicator, through the applicator, and through the nearby tissue. A layer of ice is formed on the applicator when cooled below zero degrees Celsius. Continued cooling causes the further formation or ice resulting in a larger layer of ice. The temperature measurement from within the applicator indicates the temperature within the applicator but does not directly reflect the formation of ice around the applicator. It is the formation of ice that has an effect on the surrounding tissue. Cooling a target tissue to achieve a formation of ice in only the desired region and for a known amount of time is important to achieve the intended effects. Inadequate formation of ice may result in a treating an insufficient quantity of tissue. An excessive formation of ice may result in a detrimental and unintentional impact on tissues that do not require treatment. Assessing the formation of the ice layer on the outside of an applicator and the surrounding tissue is helpful to achieve reliably repeatable and predictable results to the use of cryogenics within the body.

The layer of ice that grows on the outside surface of the applicator may serve as an insulator, therefore, the temperature measurement within the applicator may not accurately reflect the conditions that result in the formation of ice around the applicator. U.S. Publication 2008/0200829 to Abboud, et al. describes methods and systems for determining ice coverage of a catheter tip. U.S. Pat. No. 7,070,594 issued to Sherman describes systems and methods for assessing the formation of an ice ball during a cryoablation procedure. The measured applicator temperature has been used to gather empirical evidence in the treatment of tissue. What is needed is a system and method to regulate the formation of ice on a cryogenic applicator within the body of a patient.

### SUMMARY

Cooling an applicator within the body may be used for the beneficial medical effects of modifying the properties of a tissue. Measuring the temperature of the applicator may guide achieving the intended medical effect, however, the usefulness of measurement is compromised by large temperature gradients created in such an application. The formation of an ice layer around the applicator leads to the intended effect but also serves to prevent measuring critical tissue temperatures where any effect is unintended. Exemplary embodiments provide for the measurement from an ice sensor on or near the applicator. An exemplary embodiment utilizes electrical impedance from an electrode to regulate the formation of the ice around the applicator.

### BRIEF DESCRIPTION OF THE DRAWINGS

FIG. 1 is a schematic of a system to detect and regulate formation of ice in a patient.

FIG. 2 is a perspective view of the distal end of a catheter with an inflatable thermal member and a plurality of ice sensors.

FIG. 3 is a perspective view of the distal end of a catheter with an inflatable thermal member and a plurality of ice sensors disposed on said thermal member.

FIG. 4 is a conceptual diagram of an array of ice sensors and an area to be frozen.

FIG. 5 is a conceptual diagram of a cross-section of a patient with a catheter in the patient's heart and a catheter in an esophagus of the patient.

FIG. 6 is a flow diagram of a method to detect ice.

FIG. 7 is a flow diagram of a method to regulate formation of ice.

FIG. 8 is a graph characterizing the method of FIG. 7.

FIG. 9 is a flow diagram of a method to regulate cooling and active warming of a thermal member.

FIG. 10. is a graph characterizing the method of FIG. 9.

FIG. 11 is a flow diagram of a method to regulate cooling and active warming of a thermal member.

FIG. 12 is a graph characterizing the method of FIG. 11.

FIG. 13 is a flow diagram of a method to create desired conditions for a plurality of ice sensors.

FIG. 14 is a flow diagram of a method to measure an extent of ice formation.

FIG. 15 is a flow diagram of a method to regulate formation of ice based on a measured extent of ice formation.

FIG. 16 is a flow diagram of a method to regulate formation of ice based on a measured extent of ice formation.

FIG. 17 is a flow diagram of a method to form ice and to actively warm.

FIG. 18 is a flow diagram of a method to regulate the forming of ice and to regulate active warming.

### DETAILED DESCRIPTION OF THE INVENTION

Cryogenic cooling of tissue is used to change tissue properties in a beneficial manner to achieve desirable outcomes for patients with a variety of maladies. An exemplary application is the therapeutic use of cryogenic techniques for the modification of tissue within the heart of a patient for controlling or eliminating cardiac arrhythmias. During the application of the therapy, it is important that the operator receive feedback not only on the efficacy of the applied therapy, but also be alerted to undesired effects so the therapy can be modified or terminated. The application of cryogenics to tissue causes freezing of the tissue and forms ice on the tissue. A location of an application, an extent of the freezing and a duration of application are important variables in the delivery of cryogenic therapy that may contribute to therapeutic efficacy as well as produce undesired effects. Apparatus and methods are presented for assessing and regulating the formation of ice on a tissue.

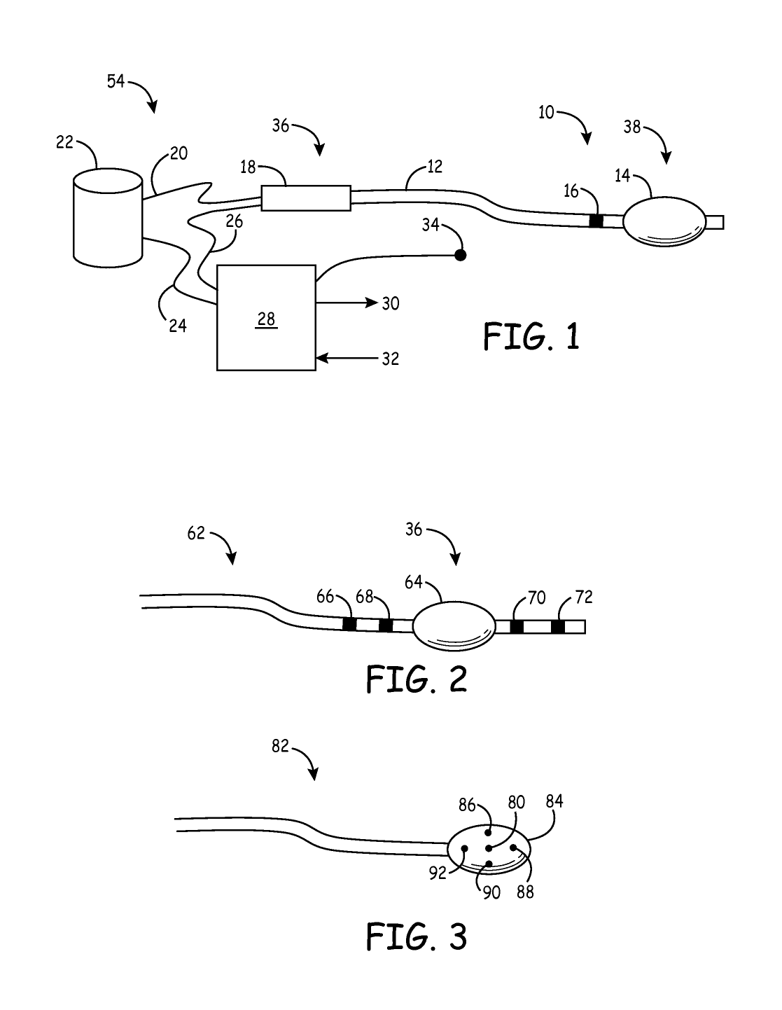

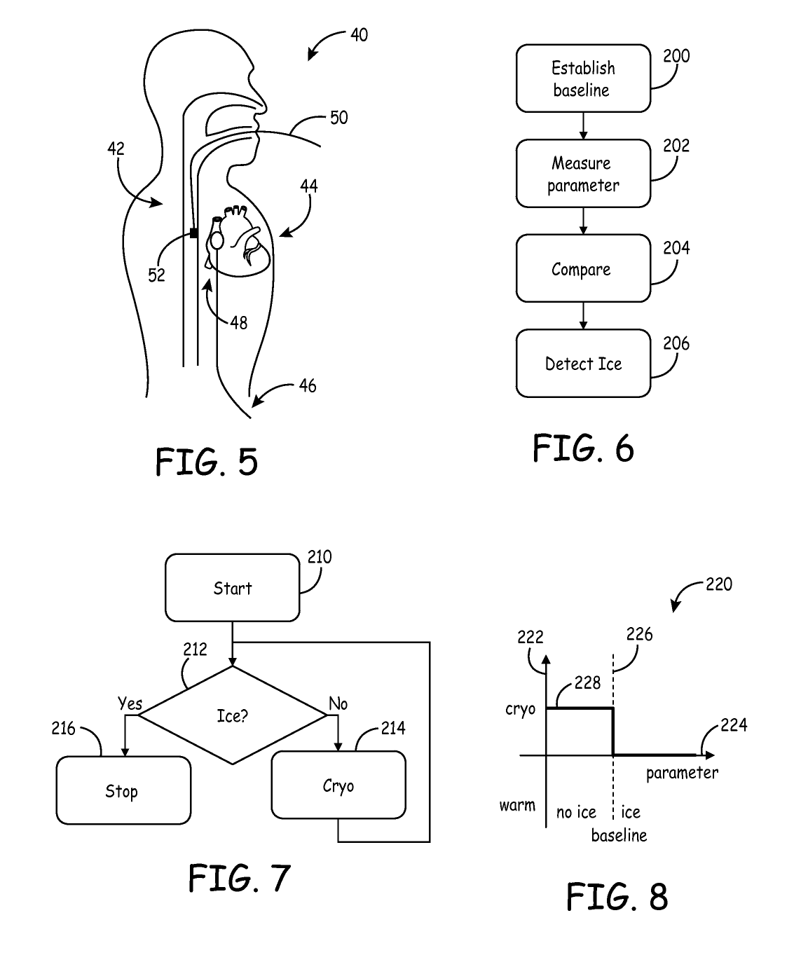

Turning to the drawings, FIG. 1 shows ice detection system 54 including catheter 10, adapted to apply cryogenic therapy to a tissue within patient 40 (FIG. 5). Catheter 10 has distal end 38 and proximal end 36. Catheter 10 includes elongate member 12, thermal member 14, electrode 16 and connector 18. Thermal member 14 and electrode 16 are located at distal end 38 of catheter 10. Connector 18 is located on proximal end 36 of catheter 10. Elongate member 12 must be of sufficient length to permit introduction into the patient from a convenient and medically safe access to a vasculature of patient 40 that extends to a target organ. The exemplary target organ described is heart 44 of patient 40. Access may be obtained from locations about the body such as the arm, the groin, the leg, the chest or other location that permits access to a desired blood vessel that leads to the heart. Elongate member 12 must be adequately flexible to permit navigation of tortuous paths to the target organ yet must also be of sufficient rigidity to permit remote manipulation by an operator. Elongate member 12 may be constructed of soft, flexible materials such as silicone rubber or various elastomers. Catheter 10 may be selected from a variety of articles that are available commercially and known in the art such as a catheter, a catheter with a cryogenic member, a basket catheter, a balloon catheter, an ablation catheter, a cardiac pacing lead, a cardiac defibrillation lead, a mapping catheter, an electrophysiology catheter, a sheath, a guidewire or an introducer.

Thermal member 14 is illustrated as an inflated balloon and an alternative construction of thermal member 14 is described below. Cold gas or liquid may be introduced and circulated through the balloon via various lumens (not shown) in elongate member 12 known in the art. These lumens may be utilized for transmission of a fluid supply and to exhaust or scavenge fluid from thermal member 14. Catheter 10 may be introduced and positioned within the body and within the heart via techniques known in the art. Catheter 10 may also include mechanisms for directing and steering within the body of patient 40. Such mechanisms may include pull-wires, push wires, cables, stylets and other mechanisms known in the art. Once positioned against heart tissue where it is desired to apply a cryogenic therapy, the balloon, thermal member 14, may be inflated and the cryogenic therapy begun by an introduction and circulation of cold gas or liquid. Warm gas or liquid may also be introduced and circulated as described below.

Elongate member 12 contains channels (not shown) for thermal communication with thermal member 14 as known in the art. The channels extend from connector 18 to thermal member 14. A channel may be included to allow the transmission of a gas or liquid to thermal member 14 and a second channel may be included for a return or evacuation of said gas or liquid from thermal member 14. The gas or liquid transmitted to thermal member 14 may be heated, cooled or at an ambient temperature. Additional channels may be provided for the transmission and return of a gas or liquid.

In an alternative embodiment, thermal member 14 may be rigid or semi-rigid and may be of the same diameter as elongate body 12. If of the same diameter, thermal member 14 would be an iso-diametric element on elongate member 12. Thermal member 14 may be constructed of metal, ceramic or other like material.

Electrode 16 is used as an ice sensor in FIG. 1, however, a variety of sensors other than an electrode may be employed to detect ice about a sensor. An ice sensor may utilize various properties such as optical, electrical, chemical, biologic, physical or utilize other principles to detect ice. For example, when illuminated by visible light, ice is white whereas, perfused tissues of the body are generally red so an optical sensor detecting the color of reflected or transmitted light could be the basis for sensing the presence of ice. A physical sensor might employ a small mechanical device such as a MEMS (microelectromechanical systems) sensor to deflect and detect rigidity of tissues surrounding a sensor, a rigid surface being an indication of ice having formed about the sensor.

While a variety of sensors might be employed, an embodiment based on electrical impedance is described. Electrode 16 is used to detect the formation of ice about electrode 16. Electrical impedance measured through electrode 16, illustrated on elongate member 12, reveals the formation of ice about electrode 16. Communication to electrode 16 is via connections through or on elongate member 12. Electrode 16 is connected to the proximal end of elongate member 12 and connector 18 via an insulated wire contained within elongate member 12. The insulated wire (not shown) connects electrode 16 and connector 18. Connector 18 is attached to proximal end of elongate member 12. Connector 18 includes an electrical connection for electrode 16, fluidic connection(s) for thermal member 14, and may include a rigid or semi-rigid elongate housing to serve as a handle for operator manipulation of catheter 10. Strain relief is provided to protect the electrical and fluidic connections. Connector 18 may incorporate operator controls for actuation of catheter 10 mechanisms that allow manipulation of catheter 10 while in the body of patient 40.

Ice detection unit 28 is electrically connected via cable 26 to catheter 10 via connector 18, to thermal source 22 via cable 24 and to electrode 34, an electrode that is in or on the body of patient 40 (see FIG. 5). Ice detection unit 28 has electrical output 30 which may be connected to a variety of output devices such as a display monitor, a printer, a data communications device or the like. Ice detection unit 28 has electrical input 32 which may be connected to a variety of input devices such as a keyboard, a data communications device, a pointing device, or the like. Ice detection unit 28 may include a processor, a memory, control/interface circuitry and measurement circuitry to perform various measurements and operations described below. Ice detection unit 28 may store and subsequently report or display various measurements such as the impedance measured through electrode 34 or data from a process such as a process to detect the formation of ice about an electrode. Various operations described below may be performed with an application specific integrated circuit (ASIC) and or with a machine requiring coded instructions. The measurements performed by ice detection unit 28 include measuring an impedance through electrodes 16, 34. The operations include control and regulation of thermal source 22 which may be accomplished with an on/off controller, described below, a proportional controller, also described below, a time based controller, a PID (proportional integral derivative) controller or regulated as directed by a user. A time based controller provides a thermal communication to a thermal member for a pre-determined amount of time. PID controllers are used in industrial control systems and can be implemented in ice detection unit 28 as described above. Ice detection unit 28 may store the impedance measurements, baseline values and the detection or sensing of ice about electrode 16.

Thermal source 22 is fluidly connected to thermal member 14 via connector 18 and electrically coupled to ice detection unit 28 via cable 24. Thermal source 22 is controlled via cable 24 and ice detection unit 28. Ice detection unit 28 provides signals to thermal source 22 via cable 24 including a signal to cool or not to cool. Other embodiments, described below, provide additional signals including a signal to warm or not to warm, a signal of a magnitude to cool and a signal of a magnitude to warm. The signals are electric and may be analog or digital or combinations of analog and digital signals. Thermal source 22 supplies a cryogenic gas or liquid to thermal member 14 via fluid connection 20, connector 18 and the channels within elongate member 12 between connector 18 and thermal member 14. Thermal source 22 also supplies a warm gas or liquid to thermal member 14 via the same fluid connection or via an additional fluid connection (not shown). That is, thermal source 22 can warm or cool catheter 10 under the control of ice detection unit 28. Although shown with one common fluid connection between thermal source 22 and connector 18, separate connections for warm and cold as well as for transmission and reception of the gas or fluid may be incorporated. After a dose of cryogenic therapy is applied to a tissue, cessation of cryogenic communication with thermal member 14 causes the cooled tissues to warm. This method of re-warming is passive warming. Providing a warm gas or liquid to thermal member 14 is active warming and will cause the tissues to warm more quickly. Such communication of a warm gas or liquid for active warming can be employed if sensing ice about catheter 10.

Ice detection unit 28 measures an impedance through electrode 16 to determine whether there is a formation of ice about electrode 16. The impedance is measured through electrode 16 and electrode 34. Electrode 34 may be located on catheter 10, on a separate catheter within patient 40, on the body surface of the patient or elsewhere within the patient. If located on catheter 10, electrode 14 must be sufficiently distant from thermal member 14 and electrode 16 in order that the ice forming effects of thermal member 14 are measured singularly by electrode 16 and not confounded by ice formation about electrode 16. A measured impedance through electrode 16 will be low when electrode 16 is within the body and no cryogenic cooling has been initiated. If electrode 16 is within the blood stream, the measured impedance will be in the order of several hundred ohms, typically about 500 ohms. If thermal member 14 is cryogenically cooled and if ice forms about electrode 16, the measured impedance through electrode 16 will rise, typically to about 2000 ohms. The rise in measured impedance forms the basis for the detection of ice about electrode 16. When the application of cryogenic cooling is terminated and when ice that forms around electrode 16 dissipates, the impedance will return approximately to values measured before the application of cryogenic cooling.

Cessation of cryogenic cooling allows passive warming of the affected tissues by virtue of the relatively large mass of the core of the body normally at a temperature of +37 degrees Celsius and the circulation of warm blood through the blood vessels. Circulating warm fluid to thermal member 14, active warming, can speed re-warming and can be used in the regulation of tissue temperature during the application of cryogenic therapy.

Electrode 16 is a single electrode disposed on distal end 38 of catheter 10. In FIG. 2, multiple ice sensors, electrodes 66, 68, 70, 72, are disposed on the distal end of elongate member 62, similar to and corresponding to the embodiment of FIG. 1. Elongate member 62 corresponds to elongate member 12 of FIG. 1 and thermal member 64, illustrated as an inflated balloon, corresponds to thermal member 14 of FIG. 1. Electrodes 66, 68, 70, 72 are disposed around thermal member 64, each similar to and corresponding to thermal member 14 in FIG. 1, and connected to an ice detection unit (not shown) corresponding to ice detection unit 28 of FIG. 1. The corresponding ice detection unit would correspond to ice detection unit 28, however, adapted to accommodate connection to, measure impedance though each of four electrodes, 66, 68, 70, 72, compare each impedance to each baseline impedance and sense ice about each electrode based on the comparisons. As impedance measured from electrode 16 of FIG. 1 reveals the presence of ice about electrode 16, impedance measured through each of the four electrodes provides information regarding formation of ice about each of the electrodes in FIG. 2. By sampling the presence of ice at various physical locations, the locations of the four electrodes, the extent of ice formation is determined. Having a plurality of ice sensors arranged on elongate member 62, learning the arrangement of the ice sensors, communicating with each ice sensor, measuring impedance through each ice sensor, and sensing ice about each ice sensor allows ice detection unit 28 to detect the extent of ice about elongate member 62 based on the sensing of ice about each sensor and the arrangement of the sensors. Ice detection unit 28 may then store, display or report the detected extent of ice.

Upon cryogenic cooling of thermal member 64, illustrated as an inflated balloon in FIG. 2, ice spreads around thermal member 64 and towards electrodes 68, 70. With continued application of cryogenic cooling to thermal member 64, the formation of ice may extend further about electrodes 66, 72 but not necessarily symmetrically about thermal member 64. The information regarding formation of ice about each of the four electrodes 66, 68, 70, 72, reveals the extent of ice formation about elongate member 62. As illustrated in FIG. 2, four electrodes are disposed around thermal member 64, however, the system need not be limited to four as more electrodes could be employed and they need not necessarily be disposed in a symmetric pattern around thermal member 64. The electrodes that are utilized as ice sensors may be located on a second elongate member (discussed below and in FIG. 5) or they may be positioned within the body by techniques other than location on an elongate member. For example, if the patient is undergoing a surgical procedure, electrodes could be placed on the outside of the heart, connected to ice detection unit 28, and be employed as ice sensors.

In FIG. 3, elongate member 82 corresponds to elongate members 12, 62 (see FIGS. 1,2). Similarly, thermal member 84, corresponds to thermal members 14, 64. Electrodes 80, 86, 88, 90, 92 disposed about thermal member 84, serve as ice sensors on the surface of thermal member 84, illustrated as an inflated balloon. Flexible wire connections from each of electrodes 80, 86, 88, 90, 92 are routed through or on elongate member 82 to a corresponding connector, cable and ice detection unit (not shown). In a manner similar to that described above for FIG. 2, the application of cryogenic cooling to thermal member 84 may cause ice to form about the thermal member. An impedance measured through each of the five electrodes provides information regarding formation of ice about each of the electrodes and that information reveals the extent of ice formation about thermal member 84 and elongate member 82. Such a system need not be limited to five electrodes, but is shown as five for clarity of illustration.

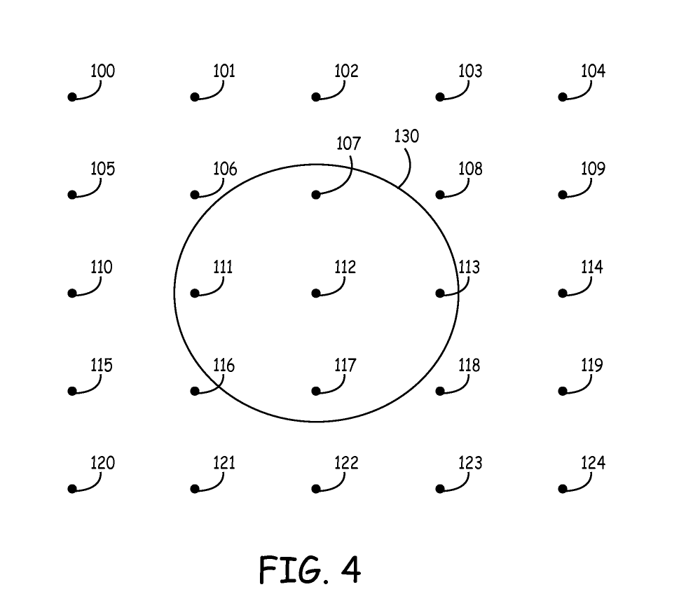

FIG. 4 illustrates a matrix of ice sensors. The matrix is illustrated as a two-dimensional array, however, it will be appreciated that this discussion is applicable to domains of three dimensions. Application of cryogenic therapy within the body may easily affect adjacent structures. For example, the application of cryogenic therapy to a portion of the heart may cause cooling and even freezing of the blood, vessels, tissue, nerves, and other organs. The extent of the cooling and the freezing of other tissues is a critical medical concern as the cooling may create transient or permanent physiologic dysfunction. Preventing deleterious effects may require moderation of the cryogenic therapy, cessation of the cryogenic therapy or cessation and rapid re-warming.

Medical planning is undertaken to identify the area or areas that are to be frozen and modified as well as the area that is to be not frozen. This anatomic and physiologic based intention of the operator to prevent injury to specific organs includes targeting an area of tissue for modification and an implicit desire to contain the effects of cryogenic cooling to the target area. FIG. 4 illustrates a planar array of ice sensors located in and around an area that is targeted to receive a cryogenic therapy. A user such as a physician, a nurse, a technician, or other individual involved in the care or treatment of the patient may undertake planning the application of the cryogenic therapy by defining area 130, the area of tissue intended to receive cryogenic treatment. Area 130, in this example, is illustrated as a circle. Area 130 need not be circular but can correspond to any suitably defined area of tissue in need of cryogenic treatment. This is the area that is to be “frozen”. By identifying area 130 as the area to be “frozen,” the user also defines that which is not within area 130 as the area that is to be “not frozen”. Ice sensors 107, 111, 112, 113, and 117 are located within area 130, the area to be “frozen”. Ice sensors 100-106, 108-110, 114-116, and 118-124 are sensors that are not within area 130 and are designated to be “not frozen”. In this figure, the array of ice sensors is presented as planar merely for visual clarity; planning for treatment within a patient will also take into account structures in three dimensions relative to the target cryogenic therapy area. The user defines a volume to be frozen. The definition may be via defining areas in a number of two dimensional views and interpolating a three dimensional surface to encompass the two dimensional views or the user may utilize various anatomic and or physiologic imaging modalities to aid in defining the volume. Alternatively, the user may specify the volume based on physical dimensions with reference to a cryogenic member or an ice detector placed or to be placed within the body. The application of cryogenic cooling to the heart is used for the correction of cardiac arrhythmias in a procedure referred to as ablation. Temporary or permanent damage to nearby organs is an undesired effect of the application of cryogenic cooling. In FIG. 5, patient 40 is shown with indwelling catheter 46 having thermal member 48. Catheter 46 has been positioned through an inferior access and is resting within heart 44. A second catheter 50 has been advanced into esophagus 42 of patient 40. Ice detector 52 on the distal end of catheter 50 is resting in esophagus 42. Ice detector 52 is adjacent to heart 44 and, more specifically, proximate thermal member 48. The potential for damage to a patient's esophagus is a concern during the application of cryogenic therapy for ablation of cardiac arrhythmias. Ice detector 52 may be utilized to monitor the formation of ice at the tissue that has the potential to be damaged. Ice detector 52 may be an electrode or may be another type of sensor, described above. Referring now to FIG. 6, a flow diagram of ice detection is described. Ice detection unit 28 detects the presence or formation of ice about electrode 14. Starting in step 200, a baseline value is established. The baseline may be established by measuring a parameter from an ice sensor before the application of a cryogenic therapy. While the ice sensor is warm, for example, while the sensor is surrounded by blood or perfused body tissue, prior to the application of cryogenic therapy, a measurement may be made at a time when it is understood ice has not been formed. Alternatively, the baseline may be based on experimental data. In the example of an electrode, an impedance measured prior to the application of cryogenic therapy could be in the range of about 300 to 500 ohms and the impedance would be about 2,000 ohms after the application of cryogenic cooling when ice has formed about the electrode. The baseline is established for use in a subsequent step to determine the presence of ice. The ice detection system distinguishes conditions where ice is present from those where ice is absent about electrode 16. Prior to the application of a cryogenic therapy ice is not present. After the application of the cryogenic therapy ice detection system 54 will issue an alert to an operator prior to an inadvertent damage of tissue from a dose of cryogenic therapy. In the example of an electrode being used as an ice detector, the baseline impedance might be about 1,000 ohms. The baseline may be established by measuring the parameter, impedance in this example, from each sensor, selecting a baseline value or a range of values for each sensor or using a pre-determined value for each sensor. To detect the presence of ice, a measured impedance from each sensor is compared to each corresponding baseline impedance.

In step 202, a parameter is measured from the ice sensor. In the example of an electrode being used as an ice sensor, the impedance is measured through the electrode. In step 204, the parameter measurement is compared to the baseline. In step 206, the detection of ice is based on the comparison in step 204. In the example of an electrode being used for the ice sensor, the measured impedance is compared to the baseline impedance established in step 200. If the measured impedance is less than the baseline impedance, ice is not present or not detected. If the measured impedance is not less than the baseline impedance, ice is present or detected. The above describes a method to detect ice including inserting a catheter into a patient, the catheter having an electrode disposed on the catheter, measuring a parameter from the electrode, the parameter being an impedance, and sensing ice about the electrode based on the measured parameter.

The sensing and detection of ice and the formation of ice is described above. The sensing and detection of the formation of ice may be used to regulate the production of ice. Proceeding to FIG. 7, the process starts in step 210 and proceeds to step 212. In step 212, the detection of ice may be conducted as exemplary described and illustrated in FIG. 6. If ice is not detected, the process proceeds to step 214 and cryogenic cooling is applied to the patient. As described above, a cryogenic gas or liquid may be communicated to thermal member 14, 16 or 84 within patient 40 to effect the cryogenic cooling. The process then continues to step 212. If ice is detected in step 212, the process continues to step 216 wherein the application of cryogenic cooling is terminated and the process ends. In this manner, the formation of ice is regulated based on the detection of ice about a sensor via ice detection unit 28.

FIG. 8 displays graph 220 characterizing ice detection system 54, described above. Abscissa 224 displays the value of a parameter measured from an ice sensor. Ordinate 222 displays the thermal communication to thermal member 14, 16 or 18. In FIG. 8, if the measured parameter is to the left of baseline 226, this indicates “no ice”. If not indicating ice, system 54 produces cryogenic cooling indicated by line 228 being in alignment with “cryo”. If the measured parameter is to the right of baseline 226, this indicates the presence of “ice”. If indicating ice, system 54 does not produce cryogenic cooling and passive warming occurs.

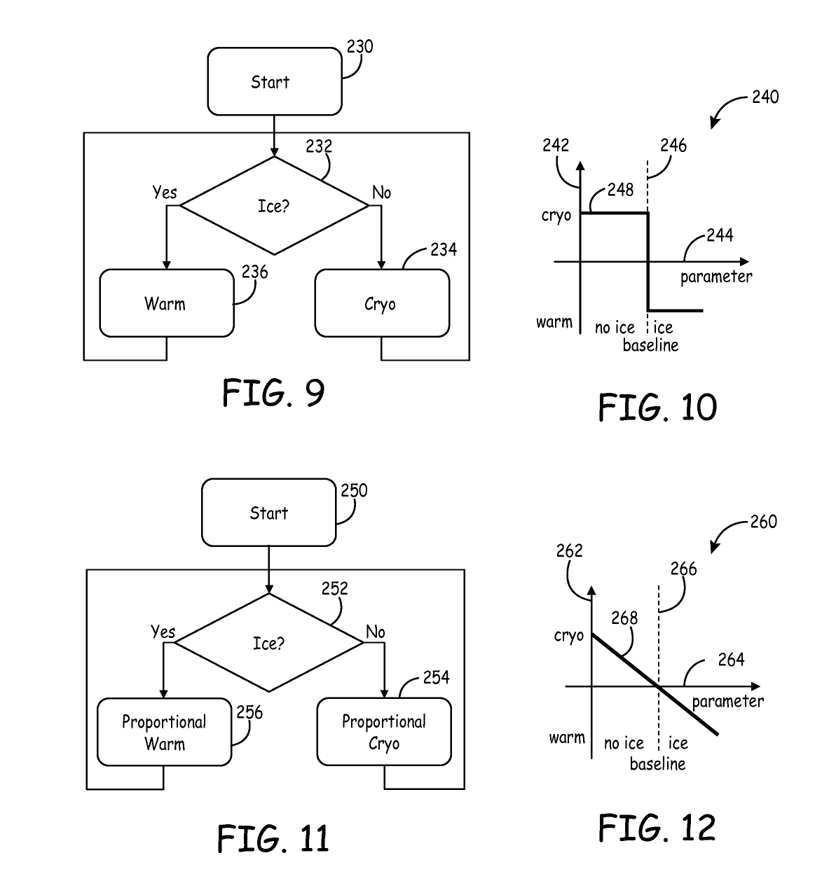

As described above, the presence of ice may be detected and the formation of ice regulated based on an ice sensor. Also as described above, thermal member 14 may be used to not only cool, but also to warm. Turning to FIG. 9, the process begins in step 230 and proceeds to step 232. In step 232, ice detection system 54 checks for the presence of ice about an ice sensor as described above. If ice is not detected about the ice sensor, indicated by “No” in step 232, the process continues to step 234 with the application of cryogenic cooling. If, in step 232, ice is detected, indicated by “Yes”, the process continues to step 236 and active warming is applied to thermal member 14. The process continues in step 232 to check for the presence of ice. If ice is, again, detected, the active warming continues. If ice is not detected, cryogenic cooling resumes in step 230. In this manner, cryogenic cooling is applied to a thermal member until ice is detected by an ice sensor. After detecting ice, active warming is applied until ice is no longer detected. Cryogenic cooling then resumes and the cycle repeats. The cycle may be interrupted at any point, based on a pre-determined time of application, a measure of desired therapeutic efficacy, or other indicators.

FIG. 10 displays graph 240 characterizing the method shown in FIG. 9 and described above. Abscissa 244 displays the value of a parameter measured from an ice sensor. Ordinate 222 displays the thermal communication to the thermal member. In FIG. 10, if the measured parameter is to the left of the baseline the system does not detect the presence of ice and system 54 applies cryogenic cooling as shown by line 248 being in alignment with “cryo”. If the measured parameter is to the right of the baseline, the system does detect the presence of ice, the system applies active warming as shown by line 248 being in alignment with “warm”. Thus, the system can regulate cryogenic cooling and active warming to thermal member 14 based on measuring a parameter from an ice sensor. FIG. 9 shows a method of active warming and cooling using an On-Off technique wherein the active warming or the cooling are either on or off with no gradation in the amount of active warming or of the cooling. Cryogenic cooling is applied or active warming is applied. The magnitude of the cooling and the magnitude of the active warming are adjusted in a binary fashion as one or the other is applied.

As described above, the presence of ice is detected and, based on measurements from an ice sensor, the formation of ice is regulated and the active warming of a member is regulated. Turning to FIG. 11, a system is described to cool and actively warm a member with incremental, rather than merely binary, gradations in the amount of active warming or cooling. The system starts in step 250 and proceeds to step 252 where system 54 checks for the presence of ice on an ice sensor. If ice is not detected, indicated by “No”, the process continues to step 254. In step 254, if active warming has been applied, it is terminated and an amount of cryogenic cooling is applied to thermal member 14, the amount based on a difference of the measured parameter from the ice sensor and the baseline value. If the difference is small, the amount of cooling applied to the thermal member is less than were the difference large. The process returns to step 252 to check for the presence of ice. If ice is detected, indicated by “Yes”, the process continues to step 256. In step 256, had cryogenic cooling been applied, it is terminated and an amount of active warming is applied to the thermal member, the amount based on the difference of the measured parameter from the ice sensor and the baseline value. If the difference is small, the amount of cooling applied to the thermal member is less than were the difference large. The process returns to step 252 to check for the presence of ice. The process can be interrupted at any time based on a pre-determined time of application, a measure of desired therapeutic efficacy, or other indicators.

FIG. 12 displays graph 260 characterizing the method shown in FIG. 11 and described above. Abscissa 264 displays the value of a parameter measured from an ice sensor. Ordinate 262 displays the thermal communication to the thermal member. In FIG. 12, if the measured parameter is to the left of the baseline the system does not detect the presence of ice and system 54 applies cryogenic cooling as shown by line 248 being above abscissa 264 and extending to “cryo”. If the measured parameter is to the right of the baseline, the system does detect the presence of ice, the system applies active warming as shown by line 248 being below abscissa 264 and extending to “warm”. Thus, the system can regulate cryogenic cooling and active warming to thermal member 14 based on measuring a parameter from an ice sensor. In contrast to the method of FIG. 9, the magnitude of the cooling and the magnitude of the active warming is proportional to the value of the parameter measured from the ice sensor. FIG. 11 shows a method of active warming and cooling using proportional controllers. Cryogenic cooling is applied or active warming is applied relative to the magnitude of the measurement from the sensor. The magnitudes of the cooling and the active warming are adjusted proportionally.

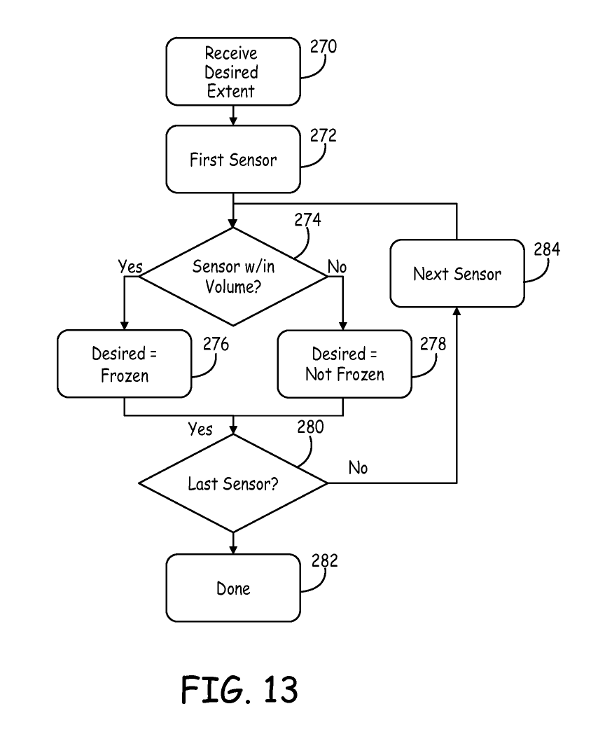

A plurality of ice sensors are placed in the body of patient 40 to detect the formation of ice during an application of cryogenic cooling. Methods are described below to establish the desired extent of ice formation and derive a specific condition for each ice sensor. Turning to FIG. 13, a method is presented based on a system with a plurality of ice sensors as exemplary shown in FIGS. 2-4. In step 270, a desired extent of ice formation is received as described above and illustrated in FIG. 4. The desired extent of ice about elongate member 62 may be established from a user input to ice detection unit 28 (see FIG. 1) or may be received from another source of data or may be a pre-determined value stored in ice detection unit 28. In step 272, the first sensor of the plurality of sensors is selected. In step 274, the sensor position is evaluated to determine whether it physically lies within a volume defined by the desired extent. If “Yes”, the sensor position is within the volume defined by the desired extent, the desired condition for that ice sensor is “Frozen” and the process continues to step 280. If “No”, the sensor position is not within the volume defined by the desired extent, the desired condition for that ice sensor is “Not Frozen” and the process continues to step 280. Step 280 checks whether the last of the plurality of sensors has been selected. If “No”, the next sensor is selected in step 284 and the process repeats in step 274. This method continues until a determination is made in step 280 that the last sensor was selected and the process terminates in step 282. The process in FIG. 13 creates a desired condition for each electrode as “frozen” or “not frozen” based on the arrangement of the electrodes and a desired extent of ice formation. The process described above evaluates each ice sensor sequentially, although, evaluations can be undertaken in any order or may be processed in parallel rather than serially. The process is presented as a serial iterative process for clarity of presentation. The process presented above may also be applied to establish a desired extent of ice about a member inserted into a patient.

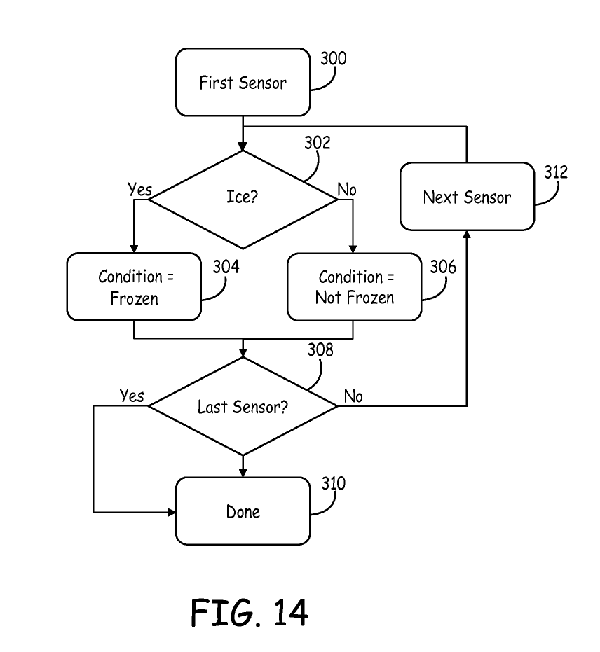

Methods described above formulate the desired extent of ice formation and derive a specific condition for each ice sensor. A method is presented below to detect the extent of ice formation. Subsequent methods will be presented to compare the detected extent with the desired extent. In FIG. 14, a first of a plurality of sensors is selected in step 300. In step 302, the system evaluates the presence of ice about the selected sensor. If “Yes”, ice is detected about the selected ice sensor, the condition for the sensor being evaluated is set in step 304 to “Frozen”. If “No”, ice is not detected about the selected ice sensor, the condition for the sensor being evaluated is set in step 306 to “Not Frozen”. Steps 304 and 306 lead to step 308 where an evaluation is performed as to whether the selected sensor is the last sensor. If “No”, the selected sensor is not the last of the plurality of sensors, the process continues to step 312 to select a next sensor and proceed to step 302 for the evaluation of the presence of ice about the selected ice sensor. This iterative process repeats as each ice sensor is evaluated and the detected condition noted as “Frozen” or “Not Frozen”. When the last sensor has been evaluated, the process proceeds from step 308, through “Yes”, the selected sensor is the last of the plurality of the sensors, to step 310 where the process terminates. In this manner, by detecting the condition of each ice sensor as having ice about the selected sensor, “Frozen”, or not having ice about the sensor, “Not Frozen”, the extent of ice formation is detected.

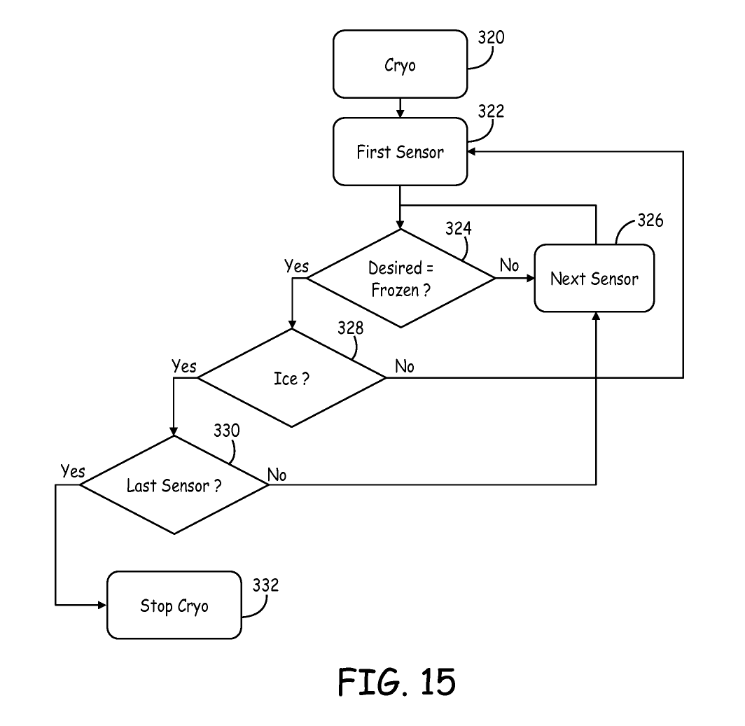

In FIG. 15, a method is presented to regulate the application of cryogenic cooling based on the extent of ice formation described above. The method of FIG. 15 applies cryogenic cooling until all sensors having a desired condition of “Frozen” have ice detected about those sensors. In step 320, cryogenic cooling is applied to a thermal member and in step 322 the first sensor is selected. In step 324, the selected ice sensor is evaluated to determine if the desired condition for the selected ice sensor is frozen. If “Yes”, the process continues to step 328 and an evaluation performed as to whether ice is detected about the selected ice sensor. If “Yes”, ice is detected about the selected ice sensor, the process continues to step 330 to determine if the selected sensor is the last of the plurality of sensors. If “Yes”, the selected sensor is the last of the plurality of sensors, the process moves to step 332 and the cryogenic cooling is stopped.

If, in step 324, the desired condition were “No”, the process proceeds to step 326 to select the next sensor and then loops back to step 324. In this manner, the sensors having a desired condition “not frozen” are not evaluated for ice about the sensors.

If, in step 328, “No”, ice is not detected about the selected ice sensor, the process returns to step 322 as the condition required for terminating the cryogenic cooling was not met. The first sensor, again, is selected and each sensor evaluated as described above. Thus, ice is formed if not sensing ice about any ice sensor having a desired condition of “Frozen” and ice is not formed if sensing ice about all sensors having a desired condition of “Frozen”. Regulation of cryogenic cooling is based on the extent of ice formation as detected by a plurality of ice sensors and cryogenic cooling is applied until all sensors that are to be “Frozen” have ice detected about them.

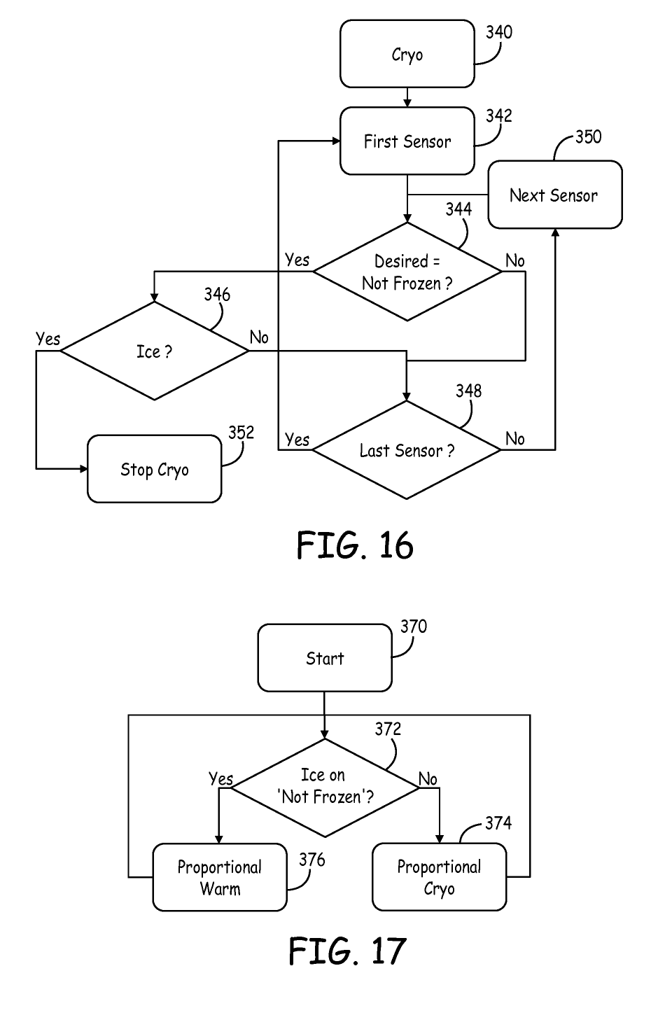

The method presented above and illustrated in FIG. 15 applies cryogenic cooling until all sensors having a desired condition of “Frozen” have ice detected about those sensors. An alternative method is presented in FIG. 16 and described below to apply cryogenic cooling until a sensor having a desired condition of “Not Frozen” has ice detected about the sensor. In step 340, cryogenic cooling is applied to a thermal member and in step 342, the first of the plurality of sensors is selected. In step 344, the selected ice sensor is evaluated to determine if the desired condition for the ice sensor is “not frozen”. If “Yes”, the process proceeds to step 346 and an evaluation made to determine whether ice is detected about the selected ice sensor. If “Yes”, ice is detected about the selected ice sensor, the cryogenic cooling is stopped in step 352. Thus, if any sensor having a desired condition of “Not Frozen” has ice about the sensor, cryogenic cooling is stopped.

In step 344, if the desired condition is not “Not Frozen”, “No”, the process proceeds to step 348. A “No” result from step 344 is equivalent to a desired condition of “Frozen”. Step 348 is reached from step 344 as just described or step 346 wherein a sensor having a condition of “Not Frozen” does not have ice detected about the sensor. Step 348 determines whether the selected sensor is the last sensor. If it is, “Yes”, the process continues to step 342 and the first sensor is, again, selected. If, in step 348, the selected sensor is not the last sensor, “No”, the process proceeds to step 350. In step 350, the selected sensor is advanced to the next sensor and the process proceeds to step 344. Thus, ice is formed if not sensing ice about all of the ice sensors having a desired condition of “Not Frozen” and ice is not formed if sensing ice about any sensor having a desired condition of “Not Frozen”. In this manner, regulation of cryogenic cooling is based on the extent of ice formation as detected by a plurality of ice sensors and cryogenic cooling is applied until one sensor having a desired condition of “Not Frozen” has ice detected about it. The forming of ice is regulated by establishing a desired extent and measuring a detected extent of ice. More specifically, the regulating is based on the detected extent and the desired extent. The methods illustrated in FIGS. 15, 16 and described above present forming ice, the detected extent being less than the desired extent and not forming ice, the detected extent not being less than the desired extent.

Methods presented above and illustrated in FIGS. 15, 16 regulate the formation of ice via starting and stopping cryogenic cooling to form ice. FIG. 17 presents a method to regulate the application of cryogenic cooling and active warming to a thermal member wherein the magnitude of the cooling and the magnitude of active warming are proportional to the extent of the formation of ice about the ice sensors. The process starts in step 370 and proceeds to step 372. In step 372, an evaluation is performed to determine whether any ice senor having a condition of “not frozen” has ice about it. If “Yes”, a sensor having a condition of “not frozen” has ice about it, the process proceeds to step 376. In step 376, a number of ice sensors having a desired condition of “not frozen” and having ice detected about the ice sensor is counted. Cryogenic cooling is stopped, had it been applied, and active warming is applied to the thermal member, the magnitude of the active warming proportional to the number counted. The process continues to step 372.

If “No”, a sensor having a condition of “not frozen” does not have ice about it, the process proceeds to step 374. In step 374, a number of ice sensors having a desired condition of “frozen” and having ice about it is counted. Active warming is stopped, had it been applied, cryogenic cooling is applied to the thermal member, the magnitude of the cryogenic cooling proportional to the number counted. The process continues to step 372. This process may be terminated by a pre-determined length of time, a measure of efficacy or by an operator intervention.

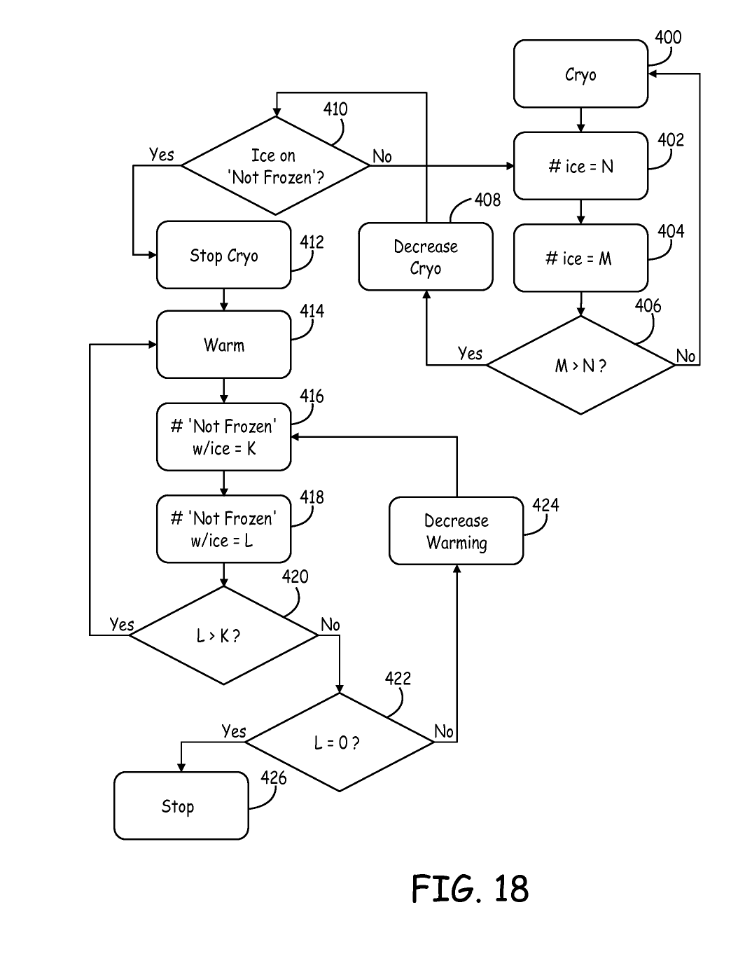

A method is presented in FIG. 18 to regulate an active warming and a cooling of a thermal member, the regulation based on a time sequential change in the number of a plurality of sensors detecting ice about the sensors. In step 400, a cryogenic cooling is applied to a thermal member. Proceeding to step 402, an evaluation is performed to count a number, N, of ice sensors having ice detected about them. Proceeding to step 404, a subsequent evaluation is performed to count a number, M, of ice sensors having ice detected about them. Note, the same evaluation is performed in steps 402, 404, however, steps 402, 404 are separated by a time interval. The time interval between the evaluations in steps 402, 404 may range from 2 to 30 seconds but is nominally 5 seconds. Proceeding to step 406, M is compared to N. If M is not greater than N, “No”, the number is not increasing and the process continues to step 400. In this situation, the number of sensors detecting ice is not changing. When the process is first begun, ice is not present on any sensor, thus, both N and M are zero. When ice begins to form and be detected around the ice sensors, N and M will be greater than zero. If M is greater than N, “Yes” in step 406, the number of ice sensors detecting ice increased over the time interval between evaluations of steps 402, 404. If “Yes”, the number is increasing and the process continues to step 408 wherein the magnitude of the cryogenic cooling is down regulated via ice detection unit 28 directing thermal source 22. The process proceeds to step 410 where all sensors with a desired condition of “not frozen” are evaluated for the presence of ice about them. If, “No”, ice is not detected about any sensor with a desired condition of “not frozen”, the process proceeds to step 402. In this manner, as a number of ice sensors detecting ice and having a desired condition of “frozen” increases, the magnitude of the cryogenic cooling will sequentially decrease. The method described above counts the number of sensors sensing ice about each sensor and then decreases a rate of forming ice if the number is increasing and does not decrease the rate of forming ice if the number is not increasing. If the number is increasing the extent of the ice formation is increasing. If the number is not increasing, the extent of the ice formation is not increasing. The method described decreases the rate of forming ice if the detected extent is increasing and the rate of forming ice is not decreased if the detected extent is not increasing.

In step 410, if a single ice sensor with a desired condition of “not frozen” has ice detected about it, the process proceeds to step 412. In step 412, cryogenic cooling is terminated and the process proceeds to step 414. In step 414, ice detection unit 28 directs thermal source 22 to begin active warming the thermal member. In this manner, active warming is performed if sensing ice about the ice sensors. Proceeding to step 416, an evaluation of the extent of the ice formation is performed to count a number, K, of ice sensors having a desired condition of “not frozen and having ice detected about them. Proceeding to step 418, a subsequent evaluation is performed to count a number, L, of ice sensors having a desired condition of “not frozen” and having ice detected about them. Note, the same evaluation is performed in steps 416, 418, however, steps 416, 418 are separated by a time interval. The time interval between the evaluations in steps 416, 418 may range from 2 to 30 seconds but is nominally 5 seconds and is not necessarily the same as the time interval between steps 402, 404.

Proceeding to step 420, L is compared to K. If L is not greater than K, “No”, the process continues to step 422. If L is not greater than K, the number of sensors having a desired condition of “Not Frozen” and detecting ice is not increasing; if L is not greater than K, the extent of ice formation is not increasing. In step 422, a decision is based on the number L, the result of step 418. If L equals zero, “Yes”, the process continues to and terminates in step 426. If L is not equal zero, “No”, the process proceeds to step 424, wherein the magnitude of the active warming is down regulated via ice detection unit 28 directing thermal source 22. The process continues to step 416. In this manner, as a number of ice sensors detecting ice and having a desired condition of “Not Frozen” does not increase, the magnitude of the active warming will sequentially decrease. Counting the number of sensors having a desired condition of “Not Frozen” evaluates the detected extent of the ice formation. The method described above regulates the active warming based on the detected extent. If a sensor having a desired condition of ‘Not Frozen’ has ice detected about the sensor, the detected extent is greater than the desired extent since the “desire” is for that sensor to not have ice about it.

In step 420, if L is greater than K, the number of sensors having a desired condition of “Not Frozen” is increasing and the extent of ice formation is increasing. The process proceeds to step 414 and active warming is continued at the initial level.

A system and method are presented above to regulate the forming of ice within a patient by inserting an elongate body into the patient, an ice sensor disposed on the elongate body, and regulating the forming based on a measurement from the ice sensor. The use of an electrode as an ice sensor is described as is inserting a second elongate member into the patient, a thermal member disposed on the second elongate member. The system includes an ice detection unit communicating with the sensor; measuring a parameter from the sensor; and a thermal source communicating a coolant with a thermal member. The ice detection unit regulates the coolant communication based on the measured parameter from the ice sensor.

We claim:

1. A method of controlling a forming of ice within a patient, comprising:
inserting a member into the patient, a plurality of electrodes disposed on the member and the member in fluid communication with a cryogenic cooling source;
defining a baseline impedance for each electrode;
measuring an impedance through each electrode;
comparing the corresponding measured and baseline impedances for each electrode;
assigning a desired condition of frozen or not frozen for each electrode;
sensing a condition about each electrode of frozen or not frozen based on the comparison of the corresponding measured and baseline impedances;
comparing the corresponding sensed and desired conditions for each electrode; and
regulating the forming of ice via the cryogenic cooling source responsive to the comparisons of the corresponding sensed and desired conditions.

2. The method of claim 1 further comprising:
forming ice if an electrode with a desired condition of frozen has a sensed condition of not frozen; or
forming ice if each electrode with a desired condition of not frozen has a sensed condition of not frozen; or
not forming ice if each electrode with a desired condition of frozen has a sensed condition of frozen; or
not forming ice if an electrode with a desired condition of not frozen has a sensed condition of frozen.

3. The method of claim 1, wherein the regulating comprises:
forming ice followed by not forming ice based on the comparisons of the corresponding sensed and desired conditions for each electrode.

4. The method of claim 1, further comprising:
measuring the impedances with an AC signal of about 20 KHz.

5. The method of claim 1, further comprising:
communicating the desired conditions and the sensed conditions.

6. The method of claim 1, wherein the member is a catheter, a catheter with a cryogenic member, a basket catheter, a balloon catheter, an ablation catheter, a cardiac pacing lead, a cardiac defibrillation lead, a mapping catheter, an electrophysiology catheter, a sheath, a guidewire or an introducer.

7. The method of claim 1, further comprising:
defining a baseline impedance for an electrode via at least one of:
establishing a baseline impedance value of about 1000 ohms;
receiving an input from a user; and
measuring an impedance.

8. The method of claim 1 wherein the regulating is via an on/off controller, a proportional controller, a time-based controller, a PID controller or via a user.

9. The method of claim 1 further comprising the step of:
detecting an extent of ice about the member based on the comparisons of the desired and the sensed conditions.

10. The method of claim 9 further comprising:
storing, reporting or displaying the detected extent.

11. A system for controlling a formation of ice within a patient comprising:
an elongate body;
a plurality of electrodes and a thermal member disposed on the elongate body;
a cryogenic cooling source in fluid communication with the thermal member;
an ice detection unit in communication with the cryogenic cooling source and the electrodes, the ice detection unit adapted to:
measure an impedance through each electrode, when the electrode has been placed in a patient's body;
detect an extent of ice along the elongate body based on the measured impedances;
assign a desired condition for each electrode as frozen or not froze; and
regulate the cryogenic cooling source responsive to the detected ice and the desired condition for each electrode.

12. The system of claim 11, wherein the ice detection unit adapted to define a baseline impedance for each electrode based on at least one of:
a selection by a user,
a value of 1000 ohms, and
a measured impedance.

13. The system of claim 12, wherein the regulating being adapted to:
form ice if an electrode with a desired condition of frozen has a sensed condition of not frozen; or
form ice if each electrode with a desired condition of not frozen has a sensed condition of not frozen; or
not form ice if each electrode with a desired condition of frozen has a sensed condition of frozen; or
not form ice if an electrode with a desired condition of not frozen has a sensed condition of frozen.

14. The system of claim 11, wherein the ice detection unit adapted to measure the impedances using an AC signal of about 20 KHz.

15. The system of claim 12, further comprising:
the ice detection unit being adapted to report, record or display at least one of
a measured impedance,
a baseline impedance,
a sensed ice, and
an extent of sensed ice.

16. The system of claim 11, wherein the ice detection unit comprises an on/off controller, a proportional controller, a time based controller, or a PID controller.

17. The system of claim 11 wherein the elongate member is a catheter, a catheter with a cryogenic member, a basket catheter, a balloon catheter, an ablation catheter, a lead, a cardiac pacing lead, a cardiac defibrillation lead, a mapping catheter, an electrophysiology catheter, a sheath, a guidewire or an introducer.

18. A method for regulating the formation of ice on a member within a body comprising:
means for cooling the member;
means for assigning desired ice conditions along the member;
means for detecting ice conditions along the member; and
regulating the cooling means responsive to the detected ice conditions means and the desired ice conditions means.
